(19) United States
(12) Patent Application Publication
(10) Pub. No.: US 2017/0245529 A1
(43) Pub. Date: Aug. 31, 2017

(54) NUTRITIONAL COMPOSITIONS COMPRISING AN OXIDIZABLE COMPONENT AND WATER-SOLUBLE PLANT EXTRACT

(71) Applicant: ABBOTT LABORATORIES, ABBOTT PARK, IL (US)

(72) Inventors: Megan Terp, Columbus, OH (US); Youngsuk Heo, Cumming, OH (US); Normanella Dewille, Columbus, OS (US); Paul Johns, Columbus, OH (US); Allison Atnip, Columbus, OH (US)

(21) Appl. No.: 15/515,760

(22) PCT Filed: Oct. 8, 2015
(86) PCT No.: PCT/US2015/054744

§ 371 Date: Mar. 30, 2017

Related U.S. Application Data
(60) Provisional application No. 62/061,339, filed on Oct. 8, 2014.

(51) Int. Cl. A23L 5/20; A23L 33/105; C08L 5/00; A23L 33/00; A61K 36/53; A61K 36/534; A23L 3/3472; A23L 33/115

(52) U.S. Cl. CPC A23L 5/20; A23L 3/3472; A23L 33/105; A23L 33/115; A23L 33/40; A61K 36/53; A61K 36/534; C08L 5/00; A23V 2002/00; A61K 2236/00

(57) ABSTRACT

A nutritional composition including an oxidizable component and a water-soluble plant extract is provided. The water-soluble plant extract includes rosmarinic acid and has a total phenolic content, such that a ratio of the total phenolic content to the rosmarinic acid is between about 1.1:1 to about 3.5:1. A method of reducing off-flavors and aromas in a nutritional composition having an oxidizable component is also provided.

### CROSS-REFERENCE TO RELATED APPLICATIONS

This application claims priority to and the benefit of U.S. Provisional Patent Application No. 62/061,339, filed Oct. 8, 2014, the entire content of which is incorporated by reference herein.

### FIELD

The general inventive concepts relate to nutritional compositions. More particularly, the general inventive concepts relate to nutritional compositions comprising an oxidizable component and a water-soluble plant extract, and methods of reducing off-flavors and aromas in a nutritional composition comprising an oxidizable component.

### BACKGROUND

A number of nutritional compositions are formulated with natural bioactive ingredients having various health benefits. For example, omega-3 polyunsaturated fatty acids derived from fish oil, which lower inflammation and provide cardiovascular health benefits, have been incorporated into nutritional compositions. However, a number of these natural bioactive ingredients, and unsaturated fatty acids in particular, are susceptible to oxidation. Nutritional compositions that incorporate these oxidizable ingredients may often have highly objectionable flavors and aromas that have been described as fishy, painty, or otherwise rancid due to oxidation that occurs during processing and storage.

### SUMMARY

The general inventive concepts relate to a nutritional composition comprising an oxidizable component and a water-soluble plant extract, and a method of reducing off-flavors and aromas in a nutritional composition comprising an oxidizable component. To illustrate various aspects of the general inventive concepts, several exemplary embodiments of nutritional compositions and methods are provided herein.

In one exemplary embodiment, a nutritional composition is provided. The nutritional composition includes a protein, a carbohydrate, a fat, and a water-soluble plant extract comprising rosmarinic acid and having a total phenolic content. At least one of the protein, the carbohydrate, and the fat includes an oxidizable component. A ratio of the total phenolic content to the rosmarinic acid ranges from about 1.1:1 to about 3.5:1. In one exemplary embodiment, the nutritional composition is in the form of an aqueous emulsion having a continuous aqueous phase and a discontinuous non-aqueous phase. In one exemplary embodiment, the nutritional composition is in the form of a powder, which may be reconstituted with a liquid, such as water, to provide a liquid nutritional composition.

In one exemplary embodiment, the water-soluble plant extract is a water-soluble rosemary extract. In one exemplary embodiment, the water-soluble plant extract is a water-soluble spearmint extract.

In one exemplary embodiment, the rosmarinic acid comprises from about 0.0004 wt % to about 0.04 wt % of the nutritional composition. In one exemplary embodiment, the total phenolic content is from about 10 milligrams (mg) to about 40 mg of gallic acid equivalents (GAE) per serving of the nutritional composition.

In one exemplary embodiment, the fat includes an oxidizable component, and the oxidizable component comprises an unsaturated fatty acid. In one exemplary embodiment, the fat including the oxidizable component is at least one of marine oil, algal oil, canola oil, flaxseed oil, borage oil, safflower oil, high oleic safflower oil, high gamma-linolenic acid (GLA) safflower oil, corn oil, soy oil, sunflower oil, high oleic sunflower oil, and cottonseed oil. In one exemplary embodiment, the oxidizable component comprises an omega-3 polyunsaturated fatty acid selected from the group consisting of eicosapentaenoic acid, docosahexaenoic acid, alpha-linolenic acid, and combinations thereof. In one exemplary embodiment, the oxidizable component comprises an omega-6 polyunsaturated fatty acid selected from the group consisting of gamma-linolenic acid, linoleic acid, dihomo-gamma-linolenic acid, arachidonic acid, and combinations thereof.

In one exemplary embodiment, the protein comprises from about 1 wt % to about 30 wt % of the nutritional composition, the carbohydrate comprises from about 5 wt % to about 25 wt % of the nutritional composition, and the fat comprises from about 0.5 wt % to about 20 wt % of the nutritional composition.

In one exemplary embodiment, a method of reducing off-flavors and aromas in a liquid nutritional composition is provided. The method includes: (a) preparing an aqueous protein solution comprising at least one protein and a water-soluble plant extract, wherein the water-soluble plant extract is extracted with a solvent system having an octanol-water partition coefficient of no more than 0.5; (b) preparing an aqueous carbohydrate solution comprising at least one carbohydrate; (c) preparing an oil blend comprising a fat having an oxidizable component; and (d) mixing together the aqueous protein solution, the aqueous carbohydrate solution, and the oil blend to form the liquid nutritional composition. In one exemplary embodiment, the water-soluble plant extract comprises a water-soluble rosemary extract. In one exemplary embodiment, the water-soluble plant extract comprises a water-soluble spearmint extract.

In one exemplary embodiment, the method further comprises at least one of heat treating the liquid nutritional composition and homogenizing the liquid nutritional composition.

In one exemplary embodiment of the method, the water-soluble plant extract comprises rosmarinic acid and has a total phenolic content, with a ratio of the total phenolic content to the rosmarinic acid being about 1.1:1 to about 3.5:1. In one exemplary embodiment, the rosmarinic acid is about 4 wt % to about 20 wt % of the water-soluble plant extract. In one exemplary embodiment, the solvent system used to extract the water-soluble plant extract comprises ethanol. In one exemplary embodiment, the solvent system used to extract the water-soluble plant extract comprises acetone.

In one exemplary embodiment of the method, the fat having the oxidizable component is at least one of marine oil, algal oil, canola oil, flaxseed oil, borage oil, safflower oil, high oleic safflower oil, high gamma-linolenic acid (GLA) safflower oil, corn oil, soy oil, sunflower oil, high oleic sunflower oil, and cottonseed oil. In one exemplary embodiment, the oxidizable component comprises an omega-3 polyunsaturated fatty acid selected from the group consisting of eicosapentaenoic acid, docosahexaenoic acid, alpha-linolenic acid, and combinations thereof. In one exemplary embodiment, the oxidizable component comprises an omega-6 polyunsaturated fatty acid selected from the group consisting of gamma-linolenic acid, linoleic acid, dihomo-gamma-linolenic acid, arachidonic acid, and combinations thereof.

### DETAILED DESCRIPTION

While the general inventive concepts are susceptible of embodiment in many different forms, described herein in detail are specific embodiments thereof with the understanding that the present disclosure is to be considered as an exemplification of the principles of the general inventive concepts. Accordingly, the general inventive concepts are not intended to be limited to the specific embodiments illustrated and described herein.

The terminology as set forth herein is for description of the embodiments only and should not be construed as limiting the disclosure as a whole. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

Any combination of method or process steps as used herein may be performed in any order, unless otherwise specified or clearly implied to the contrary by the context in which the referenced combination is made.

The term “nutritional composition” as used herein, unless otherwise specified, refers to nutritional products in various forms including, but not limited to, liquids, solids, semi-solids, semi-liquids, and any other nutritional food product known in the art. A nutritional composition in powder form may often be reconstituted to form a nutritional composition in liquid form.

The term “oxidizable component” as used herein, unless otherwise specified, refers to a component of at least one of a protein, a carbohydrate, and a fat that is susceptible to oxidation. In one exemplary embodiment, the fat includes an oxidizable component and the oxidizable component comprises an unsaturated fatty acid.

The term “fat” as used herein, unless otherwise specified, refers to lipids, fats, oils, and combinations thereof.

The terms “polyunsaturated fatty acid” and “PUFA” as used herein, unless otherwise specified, refer to any polyunsaturated fatty acid or source thereof, including short chain (less than 6 carbon atoms per chain), medium chain (greater than 6 carbon atoms per chain but less than or equal to 18 carbon atoms per chain), and long chain (having at least about 20 carbon atoms per chain) fatty acids having two or more points of unsaturation (i.e., two or more carbon-carbon double bonds).

The term “serving” as used herein, unless otherwise specified, refers to an amount of a nutritional composition administered, or intended to be administered, in a single administration event. In certain exemplary embodiments, the nutritional composition is packaged as a single serving. In certain exemplary embodiments, the nutritional composition is packaged in a container containing multiple servings, wherein the container bears instructions on how to separate a single serving of the nutritional composition from the bulk nutritional composition.

In one exemplary embodiment, a nutritional composition is provided. The nutritional composition includes a protein, a carbohydrate, a fat, and a water-soluble plant extract comprising rosmarinic acid and having a total phenolic content. At least one of the protein, the carbohydrate, and the fat includes an oxidizable component. A ratio of the total phenolic content to the rosmarinic acid ranges from about 1.1:1 to about 3.5:1. In one exemplary embodiment, the nutritional composition is in the form of an aqueous emulsion having a continuous aqueous phase and a discontinuous non-aqueous phase. In one exemplary embodiment, the nutritional composition is in the form of a powder, which may be reconstituted with a liquid, such as water, to provide a liquid nutritional composition.

Nutritional compositions that contain an oxidizable component, particularly an unsaturated fatty acid, are susceptible to the development of off-flavors and aromas over time. Such off-flavors and aromas are often described as fishy, painty, or otherwise rancid, and generally result from the oxidation of the oxidizable component. In the exemplary nutritional compositions and methods described herein, it was unexpectedly discovered that a water-soluble plant extract having a ratio of total phenolic content to rosmarinic acid of about 1.1:1 to about 3.5:1 was particularly effective in protecting the flavor and aroma of nutritional compositions comprising an oxidizable component. It was also unexpectedly discovered that a solvent system having an octanol-water partition coefficient of no more than 0.5 utilized in the extraction process to produce a water-soluble plant extract, such as a water-soluble rosemary extract, results in a higher total phenolic content, which can provide further protection against oxidation of an oxidizable component. In one exemplary embodiment, the flavor and aroma protection provided by a water-soluble rosemary extract was particularly unexpected because current technologies indicate that the primary active component of water-soluble rosemary extracts, rosmarinic acid (a polar, hydrophilic compound), exhibits less antioxidant activity in aqueous emulsion systems than non-polar, oil-soluble compounds, such as carnosic acid and carnosol, which are the primary active components of oil-soluble rosemary extracts.

As mentioned, the exemplary nutritional compositions include a protein, a carbohydrate, and a fat, and at least one of the protein, the carbohydrate, and the fat includes an oxidizable component. In one exemplary embodiment, the fat includes an oxidizable component and the oxidizable component comprises an unsaturated fatty acid. In one exemplary embodiment, the oxidizable component comprises a monounsaturated fatty acid (MUFA), a polyunsaturated fatty acid (PUFA), or both a MUFA and a PUFA. For example, in certain exemplary embodiments, the oxidizable component comprises a PUFA, such as an omega-3 PUFA, an omega-6 PUFA, or both an omega-3 PUFA and an omega-6 PUFA. In certain exemplary embodiments, the oxidizable component comprises an omega-3 PUFA. Exemplary omega-3 PUFAs include, but are not limited to, alpha-linolenic acid (ALA), eicosapentaenoic acid (EPA), and docosahexaenoic acid (DHA). In certain exemplary embodiments, the nutritional composition comprises about 0.005 wt % to about 5 wt % of omega-3 PUFAs. For example, in certain exemplary embodiments, the nutritional composition comprises about 0.005 wt % to about 4.75 wt %, including about 0.01 wt % to about 4.5 wt %, about 0.05 wt % to about 4 wt %, about 0.1 wt % to about 3.5 wt %, about 0.5 wt % to about 3 wt %, about 0.75 wt % to about 2.5 wt %, or about 1 wt % to about 2 wt %. In certain embodiments, the nutritional composition comprises about 2 wt % to about 5 wt % of omega-3 PUFAs. In one exemplary embodiment, the omega-3 PUFA comprises DHA. In one exemplary embodiment, the omega-3 PUFA comprises EPA and DHA. In certain exemplary embodiments, the EPA and DHA are present at a particular weight ratio relative to one another. For example, in certain embodiments, the weight ratio of EPA to DHA is about 1:5 to about 5:1, including about 1:4 to about 4:1, about 1:3 to about 3:1, or about 1:2 to about 2:1.

In certain exemplary embodiments, the oxidizable component comprises an omega-6 PUFA. Exemplary omega-6 PUFAs include, but are not limited to, gamma-linolenic acid (GLA), linoleic acid (LA), dihomo-gamma-linolenic acid (DGLA), and arachidonic acid (AA). In certain exemplary embodiments, the nutritional composition comprises about 0.005 wt % to about 7 wt % of omega-6 PUFAs. For example, in certain exemplary embodiments, the nutritional composition comprises about 0.005 wt % to about 5 wt % of omega-6 PUFAs, including about 0.01 wt % to about 4.5 wt %, about 0.05 wt % to about 4 wt %, about 0.1 wt % to about 3.5 wt %, about 0.5 wt % to about 3 wt %, about 0.75 wt % to about 2.5 wt %, or about 1 wt % to about 2 wt %. In certain exemplary embodiments, the nutritional composition comprises about 2 wt % to about 7 wt % of omega-6 PUFAs. In one exemplary embodiment, the omega-6 PUFA comprises gamma-linolenic acid.

In certain exemplary embodiments, the oxidizable component comprises an omega-3 PUFA and an omega-6 PUFA. For example, in certain exemplary embodiments, the oxidizable component comprises at least one omega-3 PUFA, such as ALA, EPA, and DHA, and at least one omega-6 PUFA, such as LA, GLA, DGLA, and AA. In one exemplary embodiment, the oxidizable component comprises DHA and GLA. In one exemplary embodiment, the oxidizable component comprises DHA, GLA, and EPA. It should be understood that when the oxidizable component comprises an unsaturated fatty acid, the oxidizable component may be provided by one source of fat or multiple (e.g., two, three, four) sources of fat.

In one exemplary embodiment, the fat including an oxidizable component is at least one of marine oil, algal oil, canola oil, flaxseed oil, borage oil, safflower oil, high oleic safflower oil, high gamma-linolenic acid (GLA) safflower oil, corn oil, soy oil, sunflower oil, high oleic sunflower oil, and cottonseed oil. These particular fat sources generally include a significant amount of unsaturated fatty acids, such as omega-3 PUFAs, omega-6 PUFAs, or both. Any combination of the preceding exemplary fats including an oxidizable component may be used in the nutritional compositions and methods described herein.

In one exemplary embodiment, the fat including an oxidizable component comprises marine oil containing an omega-3 PUFA. As used herein, the term “marine oil” refers to oil derived from the tissues of oily fish, oil derived from the livers of certain fish species (e.g., cod, coalfish, haddock), and oil derived from crustaceans (e.g., krill) and mollusks (e.g., squid). In certain exemplary embodiments, the marine oil is a fish oil. The fish oil may be provided by saltwater or fresh water fish including, but not limited to, albacore, menhaden, salmon, herring, mackerel, anchovies, and sardines. In certain exemplary embodiments, the fish oil may be a concentrated fish oil. In certain exemplary embodiments, the fish oil may be a microencapsulated fish oil. In one exemplary embodiment, the fish oil comprises EPA and DHA. In one exemplary embodiment, the fish oil comprises EPA and DHA at a weight ratio of about 1:5 to about 5:1 (EPA to DHA). For example, in certain embodiments, the weight ratio of EPA to DHA is about 1:5 to about 5:1, including about 1:4 to about 4:1, about 1:3 to about 3:1, or about 1:2 to about 2:1. As previously mentioned, omega-3 PUFAs, such as EPA and DHA from fish oil, are particularly susceptible to oxidation, and when oxidized can impart unpleasant off-flavors and aromas (e.g., fishy notes) to nutritional compositions that include these omega-3 PUFAs.

In one exemplary embodiment, the protein includes an oxidizable component and the oxidizable component is an amino acid having a side-chain that is susceptible to oxidation. For example, in certain exemplary embodiments, the protein includes an oxidizable component and the oxidizable component is at least one of methionine, tyrosine, tryptophan, histidine, and cysteine. In certain exemplary embodiments, the protein includes an oxidizable component and the oxidizable component is methionine. For nutritional compositions formulated as aqueous emulsions, the structure of the protein plays an important role in maintaining the stability of the emulsion by minimizing the interfacial tension between the oil droplets dispersed in the aqueous medium. Methionine, which has a relatively non-polar side-chain, can be oxidized to methionine sulfoxide, which has a more polar side-chain. Accordingly, the oxidation of methionine to methionine sulfoxide may disrupt the protein structure, which may affect the stability of the emulsion.

Nutritional compositions are generally formulated to have a relatively long shelf life, such as about 12 months to about 24 months. For nutritional compositions having an oxidizable component, the long shelf life further compounds the problems associated with the oxidation of the oxidizable component, such as the development of off-flavors and aromas over time. Accordingly, to provide protection against the oxidation of the oxidizable component and to reduce or otherwise mask any off-flavors and aromas, the exemplary nutritional compositions described herein comprise a water-soluble plant extract comprising rosmarinic acid and having a total phenolic content. More specifically, the exemplary nutritional compositions include a water-soluble plant extract having a ratio of total phenolic content to rosmarinic acid of about 1.1:1 to about 3.5:1, including about 1.25:1 to about 3.45:1, about 1.5:1 to about 3.4:1, about 1.75:1 to about 3.3:1, about 2:1 to about 3.25:1, about 2.1:1 to about 3.25:1, about 2.5:1 to about 3:1, and also including about 3.1:1 to about 3.3:1. Incorporating such a water-soluble plant extract into the nutritional composition results in the nutritional composition having a ratio of total phenolic content to rosmarinic acid of about 1.1:1 to about 3.5:1, including about 1.25:1 to about 3.45:1, about 1.5:1 to about 3.4:1, about 1.75:1 to about 3.3:1, about 2:1 to about 3.25:1, about 2.1:1 to about 3.25:1, about 2.5:1 to about 3:1, and also including about 3.1:1 to about 3.3:1. These particular ratio ranges of the total phenolic content to the rosmarinic acid have been found to be particularly effective for reducing or otherwise masking off-flavors and aromas that can develop over time in nutritional compositions containing an oxidizable component, particularly liquid nutritional compositions and aqueous emulsions.

In one exemplary embodiment, the water-soluble plant extract comprises a water-soluble rosemary extract. The water-soluble rosemary extract, in certain exemplary embodiments, has a rosmarinic acid content of about 4 wt % to about 20 wt % of the water-soluble rosemary extract. For example, in certain exemplary embodiments, the water-soluble rosemary extract comprises about 4 wt % to about 15 wt % rosmarinic acid, including about 4 wt % to about 10 wt %, about 4 wt % to about 6 wt %, or about 6 wt % to about 15 wt %. In certain exemplary embodiments, the water-soluble rosemary extract comprises about 10 wt % to about 20 wt % rosmarinic acid. Exemplary commercially available water-soluble rosemary extracts for use in the nutritional compositions described herein include AquaROX6™, AquaROX10™, and AquaROX15™ extracts available from Vitiva d.d. (Slovenia) and StabilEnhance® WSR D4 extract available from Naturex, Inc. (South Hackensack, N.J.).

In one exemplary embodiment, the water-soluble plant extract comprises a water-soluble spearmint extract. The water-soluble spearmint extract, in certain exemplary embodiments, has a rosmarinic acid content of about 1 wt % to about 20 wt % of the water-soluble spearmint extract. For example, in certain exemplary embodiments, the water-soluble spearmint extract comprises about 1 wt % to about 15 wt % rosmarinic acid, including about 1 wt % to about 10 wt %, about 2 wt % to about 10 wt %, or about 5 wt % to about 15 wt %. In certain exemplary embodiments, the water-soluble spearmint extract comprises about 10 wt % to about 20 wt % rosmarinic acid. An exemplary commercially available water-soluble spearmint extract suitable for use in the nutritional compositions described herein is FORTRA™ Dry water-soluble spearmint extract available from Kemin Industries, Inc. (Des Moines, Iowa).

In certain exemplary embodiments, when the nutritional composition is a liquid, the nutritional composition comprises about 0.0004 wt % to about 0.07 wt % rosmarinic acid. For example, in certain exemplary embodiments, the nutritional composition comprises about 0.001 wt % to about 0.07 wt % rosmarinic acid, including about 0.002 wt % to about 0.02 wt %, about 0.004 wt % to about 0.01 wt %, or about 0.01 wt % to about 0.04 wt % rosmarinic acid. In one exemplary embodiment, when the nutritional composition is a liquid, the nutritional composition comprises about 0.0004 wt % to about 0.04 wt % rosmarinic acid, including about 0.001 wt % to about 0.02 wt % rosmarinic acid, including about 0.002 wt % to about 0.012 wt %, about 0.004 wt % to about 0.01 wt %, or about 0.01 wt % to about 0.04 wt % rosmarinic acid. In certain exemplary embodiments, when the nutritional composition is a liquid, such as an aqueous emulsion, the concentration of rosmarinic acid present in the nutritional composition ranges from about 0.004 mg/mL to about 0.8 mg/mL, including about 0.01 mg/mL to about 0.6 mg/mL, about 0.04 mg/mL to about 0.4 mg/mL, about 0.06 to about 0.2 mg/mL, and also including about 0.08 mg/mL to about 0.1 mg/mL.

In certain exemplary embodiments, when the nutritional composition is a powder, the nutritional composition comprises about 0.002 wt % to about 0.35 wt % rosmarinic acid. For example, in certain exemplary embodiments, the powder nutritional composition comprises about 0.002 wt % to about 0.3 wt % rosmarinic acid, including about 0.01 wt % to about 0.2 wt %, about 0.05 wt % to about 0.1 wt %, or about 0.09 wt % to about 0.3 wt % rosmarinic acid.

In one exemplary embodiment, the water-soluble plant extract has a total phenolic content of about 100 mg of gallic acid equivalents (mg GAE) per gram of extract to about 360 mg GAE per gram of extract, including about 110 mg GAE per gram of extract to about 345 mg GAE per gram of extract, about 125 mg GAE per gram of extract to about 275 mg GAE per gram of extract, about 135 mg GAE per gram of extract to about 265 mg GAE per gram of extract, and also including about 200 mg GAE per gram of extract to about 350 mg GAE per gram of extract. In one exemplary embodiment, the water-soluble plant extract is a water-soluble rosemary extract having a total phenolic content of about 100 mg GAE per gram of extract to about 360 mg GAE per gram of extract, including about 110 mg GAE per gram of extract to about 345 mg GAE per gram of extract, about 125 mg GAE per gram of extract to about 275 mg GAE per gram of extract, about 135 mg GAE per gram of extract to about 265 mg GAE per gram of extract, and also including about 200 mg GAE per gram of extract to about 350 mg GAE per gram of extract. In one exemplary embodiment, the water-soluble plant extract is a water-soluble spearmint extract having a total phenolic content of about 100 mg GAE per gram of extract to about 300 mg GAE per gram of extract, including about 110 mg GAE per gram of extract to about 275 mg GAE per gram of extract, about 125 mg GAE per gram of extract to about 250 mg GAE per gram of extract, about 135 mg GAE per gram of extract to about 200 mg GAE per gram of extract, and also including about 100 mg GAE per gram of extract to about 250 mg GAE per gram of extract.

In certain exemplary embodiments, the nutritional composition has a total phenolic content of about 1 mg GAE to about 600 mg GAE per serving, wherein a serving is 237 mL. In certain exemplary embodiments, the nutritional composition has a total phenolic content of about 3 mg GAE to about 500 mg GAE per 237 mL serving, including about 50 mg GAE to about 400 mg GAE, about 100 mg GAE to about 300 mg GAE, and also including about 100 mg GAE to about 200 mg GAE per 237 mL serving of the nutritional composition. In certain exemplary embodiments, the nutritional composition has a total phenolic content of about 10 mg GAE to about 40 mg GAE per serving, wherein a serving is 237 mL, including about 13 mg GAE to about 40 mg GAE, about 20 mg GAE to about 40 mg GAE, about 24 mg GAE to about 36 mg GAE, and also including about 30 mg GAE to about 36 mg GAE per 237 mL serving of the nutritional composition.

In certain exemplary embodiments, when the nutritional composition is a liquid, such as an aqueous emulsion, the nutritional composition has a total phenolic content of about 0.01 mg GAE/mL to about 2.6 mg GAE/mL, including about 0.05 mg GAE/mL to about 2 mg GAE/mL, about 0.1 mg GAE/mL to about 1.5 mg GAE/mL, about 0.5 mg GAE/mL to about 1 mg GAE/mL, and also including 0.75 mg GAE/mL to about 0.9 mg GAE/mL. In certain exemplary embodiments, when the nutritional composition is a liquid, such as an aqueous emulsion, the nutritional composition has a total phenolic content of about 0.02 mg GAE/mL to about 0.2 mg GAE/mL, including about 0.05 mg GAE/mL to about 0.18 mg GAE/mL, about 0.08 mg GAE/mL to about 0.16 mg GAE/mL, and also including 0.1 mg GAE/mL to about 0.155 mg GAE/mL. Assays for determining total phenolic content are well known in the art. For example, total phenolic content can be determined using the Folin-Ciocalteu assay.

The exemplary nutritional compositions may be formulated as, and intended for consumption in, any known or otherwise suitable oral product form. Any solid, liquid, semi-solid, or semi-liquid product form, including combinations or variations thereof, are suitable for use herein, provided that such forms allow for safe and effective oral delivery of the ingredients as also defined herein. In one exemplary embodiment, the nutritional composition is one of a solid, a liquid, and a liquid reconstituted from a powder.

In one exemplary embodiment, the nutritional composition is a solid nutritional product. Non-limiting examples of solid nutritional products include snack and meal replacement products, including those formulated as bars; sticks; cookies, breads, cakes, or other baked goods; frozen liquids; candy; breakfast cereals; powders, granulated solids, or other particulates; snack chips or bites; frozen or retorted entrees; and so forth. In certain exemplary embodiments, when the nutritional composition is a solid, the serving size ranges from about 25 grams (g) to about 150 g.

In one exemplary embodiment, the nutritional composition is a nutritional liquid. Non-limiting examples of nutritional liquids include snack and meal replacement products, hot or cold beverages, carbonated or non-carbonated beverages, juices or other acidified beverages, milk or soy-based beverages, shakes, coffees, teas, liquid compositions designed for administration by nasogastric intubation, and so forth. Generally, the nutritional liquids are formulated as suspensions or emulsions, but the nutritional liquids can also be formulated in any other suitable form such as clear liquids, solutions, liquid gels, liquid yogurts, and so forth. In one exemplary embodiment, the nutritional composition is an aqueous emulsion (oil-in-water emulsion).

In one exemplary embodiment, the nutritional composition is a liquid reconstituted from a powder. In such embodiments, the liquid used to reconstitute the powder may be water or another aqueous fluid such as milk. Generally, the reconstitution rate for a particular powder will be specified on the product packaging or instructions such that the liquid reconstituted from the powder will provide the intended amount of nutrients or other components in a given volume of the liquid.

In certain exemplary embodiments, when the nutritional composition is a liquid, the serving ranges from about 30 mL to about 500 mL (˜1 fl oz to ˜17 fl oz), including from about 110 mL to about 500 mL (˜3.7 fl oz to ˜17 fl oz), from about 110 mL to about 417 mL (˜3.7 fl oz to ˜14 fl oz), from about 120 mL to about 500 mL (˜4 fl oz to ˜17 fl oz), from about 120 mL to about 417 mL (˜4 fl oz to ˜14 fl oz), from about 177 mL to about 417 mL (˜6 fl oz to ˜14 fl oz), from about 207 mL to about 296 mL (˜7 fl oz to ˜10 fl oz), from about 230 mL to about 245 mL (˜7.7 fl oz to ˜8.2 fl oz), from about 110 mL to about 237 mL (˜3.7 fl oz to ˜8 fl oz), from about 120 mL to about 245 mL (˜4 fl oz to ˜8.2 fl oz), from about 110 mL to about 150 mL (˜3.7 fl oz to ˜5 fl oz), and from about 120 mL to about 150 mL (˜4 fl oz to ˜5 fl oz).

The exemplary nutritional compositions described herein include a protein, a carbohydrate, and a fat, and at least one of the protein, the carbohydrate, and the fat includes an oxidizable component. A wide variety of sources and types of protein, carbohydrate, and fat can be used in the exemplary nutritional compositions described or otherwise suggested herein. In one exemplary embodiment, the nutritional composition includes about 1 g to about 30 g of protein per serving of the nutritional composition. For example, in certain exemplary embodiments, the nutritional composition includes about 2 g to about 25 g of protein per serving of the nutritional composition, including about 5 g to about 20 g, about 5 g to about 15 g, about 5 g to about 10 g, or about 15 g to about 25 g of protein per serving of the nutritional composition. In certain exemplary embodiments, the nutritional composition includes about 1 g to about 5 g of protein per serving of the nutritional composition. The various mass ranges for the protein may apply to any of the previously described forms of the nutritional composition.

Alternatively, the amount of protein in the nutritional composition may be expressed in terms of a weight percent (wt %) of the nutritional composition. For example, in one exemplary embodiment, the protein is present in an amount of about 1 wt % to about 30 wt % of the nutritional composition. In certain exemplary embodiments, the protein is present in an amount of about 1 wt % to about 25 wt % of the nutritional composition, including about 1 wt % to about 20 wt %, about 1 wt % to about 15 wt %, about 1 wt % to about 10 wt %, about 5 wt % to about 10 wt %, or about 10 wt % to about 20 wt % of the nutritional composition. In certain exemplary embodiments, the protein is present in an amount of about 1 wt % to about 5 wt % of the nutritional composition. In certain exemplary embodiments, the protein is present in an amount of about 20 wt % to about 30 wt % of the nutritional composition. The various weight percent ranges for the protein may apply to any of the previously described forms of the nutritional composition.

Various sources of protein, including one source or more than one source, may be used in the exemplary nutritional compositions described herein. For example, the source of protein may include, but is not limited to, intact, hydrolyzed, and partially hydrolyzed protein, which may be derived from any known or otherwise suitable source such as milk (e.g., casein, whey), animal (e.g., meat, fish), cereal (e.g., rice, corn), vegetable (e.g., soy, pea), and combinations thereof. The source of protein may also include a mixture of amino acids (often described as free amino acids) known for use in nutritional products or a combination of such amino acids with the intact, hydrolyzed, and partially hydrolyzed proteins described herein. The amino acids may be naturally occurring or synthetic amino acids.

More particular examples of sources of protein which may be suitable for use in the exemplary nutritional compositions described herein include, but are not limited to, whey protein concentrates, whey protein isolates, whey protein hydrolysates, acid caseins, sodium caseinates, calcium caseinates, potassium caseinates, casein hydrolysates, milk protein concentrates, milk protein isolates, milk protein hydrolysates, nonfat dry milk, condensed skim milk, soy protein concentrates, soy protein isolates, soy protein hydrolysates, pea protein concentrates, pea protein isolates, pea protein hydrolysates, collagen proteins, collagen protein isolates, rice proteins, potato proteins, earthworm proteins, insect proteins, and combinations thereof. The nutritional compositions can include any individual source of protein or a combination of two or more of the various sources of protein listed above.

In one exemplary embodiment, the nutritional composition includes about 10 g to about 110 g of carbohydrate per serving of the nutritional composition. For example, in certain exemplary embodiments, the nutritional composition includes about 25 g to about 90 g of carbohydrate per serving of the nutritional composition, including about 30 g to about 85 g, about 35 g to about 75 g, or about 40 g to about 60 g of carbohydrate per serving of the nutritional composition. In certain exemplary embodiments, the nutritional composition includes about 10 g to about 20 g of carbohydrate per serving of the nutritional composition. The various mass ranges for the carbohydrate may apply to any of the previously described forms of the nutritional composition.

Alternatively, the amount of carbohydrate in the nutritional composition may be expressed in terms of a weight percent (wt %) of the nutritional composition. For example, in one exemplary embodiment, the carbohydrate is present in an amount of about 5 wt % to about 75 wt % of the nutritional composition. In certain exemplary embodiments, the carbohydrate is present in an amount of about 5 wt % to about 70 wt % of the nutritional composition, including about 5 wt % to about 65 wt %, about 10 wt % to about 65 wt %, about 20 wt % to about 65 wt %, about 30 wt % to about 65 wt %, about 40 wt % to about 65 wt %, or about 15 wt % to about 25 wt % of the nutritional composition. The various weight percent ranges for the carbohydrates may apply to any of the previously described forms of the nutritional composition.

Carbohydrates suitable for use in the exemplary nutritional compositions described herein may be simple, complex, variations, or combinations thereof. Various sources of carbohydrate may be used so long as the source is suitable for use in a nutritional composition and is otherwise compatible with any other selected ingredients or features present in the nutritional composition. Non-limiting examples of a source of carbohydrate suitable for use in the exemplary nutritional compositions described herein include maltodextrin, hydrolyzed starch, glucose polymers, corn syrup, corn syrup solids, rice-derived carbohydrates, sucrose, glucose, fructose, lactose, high fructose corn syrup, honey, sugar alcohols, isomaltulose, sucromalt, pullulan, potato starch, corn starch, fructooligosaccharides, galactooligosaccharides, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, and combinations thereof. The exemplary nutritional compositions may include any individual source of carbohydrate or a combination two or more of the various sources of carbohydrate listed above.

In one exemplary embodiment, the nutritional composition includes about 0.5 g to about 50 g of fat per serving of the nutritional composition. For example, in certain exemplary embodiments, the nutritional composition includes about 1 g to about 46 g of fat per serving of the nutritional composition, including about 1.25 g to about 40 g, about 5 g to about 35 g, about 10 g to about 25 g, about 15 g to about 20 g, about 1 g to about 5 g, about 20 g to about 50 g, about 30 g to about 50 g, or about 40 g to about 50 g of fat per serving of the nutritional composition. The various mass ranges for the fat may apply to any of the previously described forms of the nutritional composition.

Alternatively, the amount of fat in the nutritional composition may be expressed in terms of a weight percent (wt %) of the nutritional composition. For example, in one exemplary embodiment, the fat is present in an amount of about 0.5 wt % to about 30 wt % of the nutritional composition. In certain exemplary embodiments, the fat is present in an amount of about 1 wt % to about 30 wt % of the nutritional composition, including about 5 wt % to about 30 wt %, about 10 wt % to about 30 wt %, about 15 wt % to about 30 wt %, about 20 wt % to about 25 wt %, about 5 wt % to about 10 wt %, or about 10 wt % to about 20 wt % of the nutritional composition. The various weight percent ranges for the fat may apply to any of the previously described forms of the nutritional composition.

As previously discussed, in certain exemplary embodiments, the nutritional composition comprises a fat including an oxidizable component. Accordingly, the nutritional composition may include any one or more of the previously discussed fats that include an oxidizable component. In certain exemplary embodiments, the nutritional composition may also include a fat selected from the group consisting of coconut oil, fractionated coconut oil, medium chain triglycerides (MCT) oil, palm oil, palm kernel oil, palm olein, and combinations thereof.

The concentration and relative amounts of protein, carbohydrate, and fat in the exemplary nutritional compositions can vary considerably depending upon, for example, the specific dietary needs of the intended user or the particular formulation of the nutritional composition (e.g., oil-in-water emulsion, powder, bar). In one exemplary embodiment, when the nutritional composition is a liquid, the liquid nutritional composition comprises total protein in an amount of about 1 wt % to about 15 wt % of the nutritional composition, total carbohydrate in an amount of about 5 wt % to about 25 wt % of the nutritional composition, and total fat in an amount of about 0.5 wt % to about 12 wt % of the nutritional composition.

In certain exemplary embodiments, the amount of protein, carbohydrate, and fat in the exemplary nutritional composition may alternatively be expressed in terms of the percentage of total calories provided by a particular macronutrient, or a concentration (e.g., grams of macronutrient per 100 mL of a liquid nutritional composition), as shown below in Table 1.

TABLE 1

Embodiments

| Macronutrient | A | B | C |
|---|---|---|---|
| Protein - % of total calories | 5-40 | 10-30 | 15-25 |
| Carbohydrate - % of total calories | 10-70 | 20-60 | 40-60 |
| Fat - % of total calories | 10-65 | 10-50 | 15-35 |
| Protein - g/100 mL | 0.5-30 | 1-15 | 2-10 |
| Carbohydrate - g/100 mL | 1-40 | 4-30 | 10-20 |
| Fat - g/100 mL | 0.1-30 | 0.5-15 | 1-5 |

The exemplary nutritional compositions described herein are useful for providing supplemental, primary, or sole sources of nutrition. In certain exemplary embodiments, the nutritional compositions provide up to about 500 kcal of energy per serving of the nutritional composition, including from about 20 kcal to about 500 kcal, from about 75 kcal to about 500 kcal, from about 150 kcal to about 500 kcal, from about 250 kcal to about 500 kcal, from about 300 kcal to about 500 kcal, or from about 400 kcal to about 500 kcal per serving of the nutritional composition.

In one exemplary embodiment, when the nutritional composition is a liquid, the liquid nutritional composition has a caloric density of about 0.5 kcal/mL to about 3 kcal/mL. In certain exemplary embodiments, the liquid nutritional composition has a caloric density of about 0.5 kcal/mL to about 2.5 kcal/mL, including about 0.5 kcal/mL to about 2 kcal/mL, about 0.5 kcal/mL to about 1.5 kcal/mL, about 0.5 kcal/mL to about 1 kcal/mL, or about 0.5 kcal/mL to about 0.8 kcal/mL. In certain exemplary embodiments, the liquid nutritional composition has a caloric density of about 1 kcal/mL to about 3 kcal/mL, including about 1.5 kcal/mL to about 3 kcal/mL, about 2 kcal/mL to about 3 kcal/mL, or about 2.5 kcal/mL to about 3 kcal/mL. In one exemplary embodiment, when the nutritional composition is a liquid, the liquid nutritional composition has a pH of about 6 to about 8.

In one exemplary embodiment, the nutritional composition includes at least one antioxidant in addition to the water-soluble plant extract. For example, in certain exemplary embodiments, the nutritional composition further comprises at least one antioxidant selected from the group consisting of ascorbic acid, ascorbyl palmitate, retinyl palmitate, tocopherols, ascorbate salts, carotenoids, oil-soluble rosemary extract, and combinations thereof. Tocopherols suitable for use in the nutritional composition may be natural or synthetic and include, but are not limited to, alpha-tocopherol, d-alpha-tocopherol (RRR-alpha-tocopherol), beta-tocopherol, gamma-tocopherol, delta-tocopherol, d,l-alpha-tocopherol (All-rac-alpha-tocopherol), tocopherol acetate, and combinations thereof. The term “tocopherol” as used herein, unless otherwise specified, also includes tocotrienols. Carotenoids suitable for use in the nutritional composition include, but are not limited to, beta-carotene, lutein, lycopene, zeaxanthin, and combinations thereof. The oil-soluble rosemary extract includes carnosic acid and carnosol as active ingredients. Exemplary oil-soluble rosemary extracts include SyneROX™ extract available from Vitiva d.d. (Slovenia) and StabilEnhance® OSR extract available from Naturex, Inc. (South Hackensack, N.J.).

In one exemplary embodiment, the nutritional composition may further comprise one or more additional components that may modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth.

In one exemplary embodiment, the nutritional composition may further include vitamins or related nutrients, non-limiting examples of which include vitamin A, vitamin B₁₂, vitamin C, vitamin D, vitamin K, thiamine, riboflavin, pyridoxine, niacin, folic acid, pantothenic acid, biotin, choline, inositol, salts and derivatives thereof, and combinations thereof.

In one exemplary embodiment, the nutritional composition may further include minerals, non-limiting examples of which include calcium, phosphorus, magnesium, zinc, manganese, sodium, potassium, molybdenum, chromium, iron, copper, chloride, and combinations thereof.

As previously discussed, nutritional compositions that contain an oxidizable component, particularly an unsaturated fatty acid, are susceptible to the development of off-flavors and aromas due to oxidation of the oxidizable component during processing and/or storage. Accordingly, in one exemplary embodiment, a method of reducing off-flavors and aromas in a liquid nutritional composition comprising an oxidizable component is provided. The method includes: (a) preparing an aqueous protein slurry comprising at least one protein and a water-soluble plant extract, wherein the water-soluble plant extract is extracted with a solvent system having an octanol-water partition coefficient of no more than 0.5; (b) preparing an aqueous carbohydrate slurry comprising at least one carbohydrate; (c) preparing an oil blend comprising a fat having an oxidizable component; and (d) mixing together the aqueous protein slurry, the aqueous carbohydrate slurry, and the oil blend to form the liquid nutritional composition. Any of the exemplary liquid nutritional compositions described herein may be prepared in accordance with this exemplary method and may incorporate any of the previously described ingredients.

The aqueous protein slurry may be prepared, according to one exemplary embodiment, by heating a predetermined amount of water to a temperature of about 60° C. to about 70° C. and mixing in a predetermined amount of protein and a predetermined amount of water-soluble plant extract. In one exemplary embodiment, the water-soluble plant extract comprises a water-soluble rosemary extract. In other exemplary embodiments, the water-soluble plant extract comprises a water-soluble spearmint extract. While not wishing to be bound by theory, it is believed that the water-soluble plant extract (e.g., water-soluble rosemary extract, water-soluble spearmint extract) and the protein interact and provide enhanced protection against oxidation in an aqueous emulsion by preferentially accumulating at the active oil-water interfaces. The protein may be one or more of the proteins previously described herein, such as sodium caseinate, hydrolyzed sodium caseinate, and whey protein concentrate.

As mentioned, the water-soluble plant extract is extracted with a solvent system having an octanol-water partition coefficient of no more than 0.5. As used herein the term “octanol-water partition coefficient” refers to the logarithm of the molecular 1-octanol-water partition coefficient (log P), and is a measure of how hydrophobic or hydrophilic a solvent system is. While not wishing to be bound by theory, it is believed that the octanol-water partition coefficient of the solvent system utilized to extract the water-soluble plant extract affects the total phenolic content of the water-soluble plant extract, with solvent systems having a lower octanol-water partition coefficient yielding a water-soluble plant extract having a higher total phenolic content.

In one exemplary embodiment, the solvent system utilized to extract the water-soluble plant extract comprises ethanol. Ethanol has an octanol-water partition coefficient of about −0.3. An exemplary water-soluble plant extract that is extracted with ethanol is StabilEnhance® WSR D4 water-soluble rosemary extract, which is available from Naturex, Inc. (South Hackensack, N.J.). In one exemplary embodiment, the solvent system utilized to extract the water-soluble plant extract comprises acetone. Acetone has an octanol-water partition coefficient of about −0.24. An exemplary water-soluble plant extract that is extracted with acetone is AquaROX15™ water-soluble rosemary extract, which is available from Vitiva d.d. (Slovenia).

The relationship between the total phenolic content and the rosmarinic acid content of the water-soluble plant extract is important for protecting against oxidation and improving the sensory characteristics (e.g., taste, aroma) of nutritional compositions having an oxidizable component, such as polyunsaturated fatty acids. In certain exemplary embodiments of the method, the water-soluble plant extract has a ratio of total phenolic content to rosmarinic acid of about 1.1:1 to about 3.5:1, including about 1.25:1 to about 3.45:1, about 1.5:1 to about 3.4:1, about 1.75:1 to about 3.3:1, about 2:1 to about 3.25:1, about 2.1:1 to about 3.25:1, about 2.5:1 to about 3:1, and also including about 3.1:1 to about 3.3:1.

The aqueous carbohydrate slurry may be prepared, according to one exemplary embodiment, by heating a predetermined amount of water to a temperature of about 60° C. to about 75° C. and mixing in a predetermined amount of carbohydrate. The carbohydrate may be one or more of the carbohydrates described herein, such as maltodextrin, sucrose, fructooligosaccharides, soy fiber, gum arabic, gellan gum, and so forth. In addition, the aqueous carbohydrate slurry may include minerals, preservatives, stabilizers, thickeners, and so forth.

The oil blend comprising a fat having an oxidizable component may be prepared, according to one exemplary embodiment, by heating a predetermined amount of fat (including fats having an oxidizable component and, optionally, other fats) to a temperature of about 25° C. to about 35° C., and mixing in an emulsifying agent, such as lecithin. In certain exemplary embodiments, the oil blend further comprises at least one antioxidant. The fats utilized in the oil blend may be one or more of the fats described herein, such as marine oil, fractionated coconut oil, canola oil, soy oil, and so forth. The at least one antioxidant may be one or more of the antioxidants described herein, such as ascorbyl palmitate, mixed tocopherols, oil-soluble rosemary extract, and so forth. In addition, the oil blend may further comprise oil soluble vitamins including, but not limited to, vitamin A, vitamin D, vitamin E, vitamin K, and combinations thereof.

The aqueous protein slurry, the aqueous carbohydrate slurry, and the oil blend are mixed together to form the liquid nutritional composition. In one exemplary embodiment, the pH of the liquid nutritional composition may be adjusted to a desired pH range (e.g., from about 6 to about 8). In one exemplary embodiment, the liquid nutritional composition is heat treated and homogenized. For example, the heat treatment is utilized to sterilize the liquid nutritional composition, and may be performed, for example, by high-temperature short-time (“HTST”) processing or ultra-high temperature processing (“UHT”). Following heat treatment and homogenization, a water soluble vitamin solution may be added (if applicable), and the pH may be adjusted (if necessary), flavorant may be added, and any additional water may be added to adjust the solids content to the desired range. At this point, the liquid nutritional composition may optionally be packaged and sterilized according to any suitable sterilization technique (e.g., aseptic, retort, hot-fill, chemical, radiation, and filtering sterilization techniques).

It should be understood that when discussing the liquid nutritional compositions disclosed herein, the discussion is equally applicable to nutritional compositions that are produced according to the exemplary methods described herein. In other words, in certain exemplary embodiments, the exemplary methods can be viewed as methods for preparing the exemplary liquid nutritional compositions described herein. However, it should also be understood that the exemplary methods described herein can also be used to prepare nutritional compositions that vary in one or more ways from the exemplary liquid nutritional compositions described herein.

### EXAMPLES

The following examples illustrate certain exemplary embodiments of the nutritional compositions and methods described herein. The examples are given solely for the purpose of illustration and are not to be construed as limitations of the general inventive concepts, as many variations thereof are possible without departing from the spirit and scope of the general inventive concepts.

### Examples 1-3

Examples 1-3 illustrate exemplary nutritional compositions in accordance with the general inventive concepts presented herein. The nutritional compositions of Examples 1-3 are in the form of an aqueous emulsion. All ingredient amounts in Examples 1-3 are listed in Table 2 as kg per 1000 kg batch of the nutritional composition, unless otherwise indicated.

TABLE 2

| | Example 1 | Example 2 | Example 3 |
|---|---|---|---|
| | Amount | Amount | Amount |
| INGREDIENTS | (kg/1000 kg) | (kg/1000 kg) | (kg/1000 kg) |
| Water | Q.S. | Q.S. | Q.S. |
| Maltodextrin | 153.44 | 153.44 | 153.44 |
| Sodium Caseinate | 31.5 | 31.5 | 31.5 |
| Sodium Caseinate | 31.1 | 31.1 | 31.1 |
| Hydrolyzed | | | |
| Sucrose | 18.3 | 18.3 | 18.3 |
| Marine Oil | 14.4 | 14.4 | 14.4 |
| Fructooligosaccharide | 11.5 | 11.5 | 11.5 |
| Powder | | | |
| Gum Arabic | 8.5 | 8.5 | 8.5 |
| Whey Protein Concentrate | 3.8 | 3.8 | 3.8 |
| (75%) | | | |
| Medium Chain | 3.6 | 3.6 | 3.6 |
| Triglycerides (MCT) Oil | | | |
| Micronized Tricalcium | 3.5 | 3.5 | 3.5 |
| Phosphate | | | |
| Potassium Citrate | 3.3 | 3.3 | 3.3 |
| Magnesium Chloride | 2.8 | 2.8 | 2.8 |
| Sodium Citrate | 2.3 | 2.3 | 2.3 |
| Canola Oil | 2.1 | 2.1 | 2.1 |
| Natural and Artificial | 2.0 | 2.0 | 2.0 |
| Vanilla Flavor | | | |
| Soy Fiber | 1.7 | 1.7 | 1.7 |
| Soy Oil | 1.2 | 1.2 | 1.2 |
| Water-Soluble Rosemary | 3.0 | — | — |
| Extract (TPC:RA ≈ 3.21:1) | | | |
| Potassium Hydroxide | 0.900 | 0.900 | 0.900 |
| (40%) | | | |
| Lecithin | 0.897 | 0.8967 | 0.8967 |
| Ascorbic Acid | 0.790 | 0.790 | 0.790 |
| Water-Soluble Rosemary | — | 2.0 | — |
| Extract (TPC:RA ≈ 3.16:1) | | | |
| Choline Chloride | 0.551 | 0.551 | 0.551 |
| Potassium Chloride | 0.517 | 0.517 | 0.517 |
| Vitamin DEK Premix | 0.399 | 0.399 | 0.399 |
| Water-Soluble Rosemary | — | — | 1.2 |
| Extract (TPC:RA ≈ 1.21:1) | | | |
| Potassium Hydroxide | 0.305 | 0.305 | 0.305 |
| Vitamin Premix | 0.260 | 0.260 | 0.260 |
| Taurine | 0.212 | 0.212 | 0.212 |
| Vitamin E (RRR-d-alpha) | 0.145 | 0.145 | 0.145 |
| tocopheryl acetate | | | |
| Zinc Sulfate | 0.0970 | 0.0970 | 0.0970 |
| L-Carnitine | 0.0950 | 0.0950 | 0.0950 |
| Gellan Gum | 0.0600 | 0.0600 | 0.0600 |
| Ferrous Sulfate | 0.0220 | 0.0220 | 0.0220 |
| Beta Carotene (30%) | 0.0182 | 0.0182 | 0.0182 |
| Manganese Sulfate | 0.0127 | 0.0127 | 0.0127 |
| Ascorbyl Palmitate | 0.0120 | 0.0120 | 0.0120 |
| Vitamin A Palmitate | 0.0060 | 0.0060 | 0.0060 |
| Cupric Sulfate | 0.0026 | 0.0026 | 0.0026 |
| Sodium Bicarbonate | 0.0024 | 0.0024 | 0.0024 |
| Tocopherol-2 (mixed | 0.0020 | 0.0020 | 0.0020 |
| tocopherols) | | | |
| Folic Acid | 0.0016 | 0.0016 | 0.0016 |
| Chromium Chloride | 0.000450 | 0.000450 | 0.000450 |
| Sodium Molybdate | 0.000300 | 0.000300 | 0.000300 |
| Potassium Iodide | 0.000198 | 0.000198 | 0.000198 |
| Sodium Selenate | 0.000171 | 0.000171 | 0.000171 |
| Vitamin K | 0.000037 | 0.000037 | 0.000037 |

### Example 4

Examples 4 illustrates an exemplary nutritional composition in accordance with the general inventive concepts presented herein. The nutritional composition of Example 4 is in the form of a powder. All ingredient amounts in Example 4 are listed in Table 3 as kg per 1000 kg batch of the nutritional composition, unless otherwise indicated.

TABLE 3

| | Example 4 |
|---|---|
| INGREDIENTS | Amount (kg/1000 kg) |
| Maltrin M200 | 346.1 |
| Milk Protein Concentrate (80%) | 141.0 |
| Calcium Caseinate | 128.6 |
| Sucrose | 121.6 |
| Fructooligosaccharide Powder | 73.7 |
| Marine Oil | 59.3 |
| Gum Arabic | 28.7 |
| Whey Protein Concentrate | 15.6 |
| Fractionated Coconut (MCT) Oil | 14.8 |
| Soy Oil | 8.33 |
| Soy Fiber | 7.04 |
| Magnesium Sulfate | 6.88 |
| Sodium Chloride | 5.59 |
| Vanilla Coconut Flavor | 5.05 |
| Sodium Citrate | 4.81 |
| Caramel Flavor | 3.84 |
| Lecithin | 3.48 |
| Water-Soluble Rosemary Extract | 3.18 |
| (TPC:RA ≈ 3.16:1) | |
| Ascorbic Acid | 3.03 |
| Potassium Citrate | 2.94 |
| Vitamin Premix | 2.76 |
| Disodium Phosphate Duohydrate | 2.73 |
| High Oleic Sunflower Oil | 2.63 |
| Choline Chloride | 2.43 |
| Potassium Hydroxide (45%) | 2.09 |
| Cinnamon Flavor | 1.47 |
| Ascorbyl Palmitate | 1.17 |
| Taurine | 0.796 |
| Potassium Chloride | 0.577 |
| Tocopherol-2 (mixed tocopherols) | 0.556 |
| L-Carnitine | 0.373 |
| Zinc Sulfate | 0.344 |
| Ferrous Sulfate | 0.128 |
| Beta Carotene (30%) | 0.0712 |
| Manganese Sulfate | 0.0455 |
| Cupric Sulfate | 0.0108 |
| Vitamin D3 | 0.00800 |
| Chromium Chloride | 0.00169 |
| Sodium Molybdate | 0.00113 |
| Sodium Selenate | 0.000719 |
| Potassium Iodide | 0.000482 |

### Examples 5-6

Examples 5 and 6 illustrate exemplary nutritional compositions in accordance with the general inventive concepts presented herein. The nutritional compositions of Examples 5 and 6 are in the form of a powder. All ingredient amounts in Examples 5 and 6 are listed in Table 4 as kg per 1000 kg batch of the nutritional composition, unless otherwise indicated.

TABLE 4

| | Example 5 | Example 6 |
|---|---|---|
| | Amount | Amount |
| INGREDIENTS | (kg/1000 kg) | (kg/1000 kg) |
| Maltodextrin DE18 | 298.2 | 298.2 |
| Milk Protein Concentrate (80%) | 159.4 | 159.4 |
| Sucrose | 152.8 | 152.8 |
| Calcium Caseinate | 133.2 | 133.2 |
| Soy Oil | 87.5 | 87.5 |
| Marine Oil | 71.0 | 71.0 |
| Natural and Artificial Flavors | 18.739 | 18.739 |
| Potassium Citrate | 16.8 | 16.8 |
| Magnesium Phosphate Dibasic | 12.5 | 12.5 |
| Sodium Chloride | 9.04 | 9.04 |
| Sodium Citrate | 7.44 | 7.44 |
| Lecithin | 6.61 | 6.61 |
| Water-Soluble Spearmint Extract | — | 5.73 |
| (TPC:RA ≈ 2:1) | | |
| Calcium Carbonate | 4.35 | 4.35 |
| Ascorbic Acid | 3.8 | 3.8 |
| Water-Soluble Rosemary Extract | 3.18 | — |
| (TPC:RA ≈ 3.16:1) | | |
| Choline Chloride | 2.97 | 2.97 |
| Potassium Hydroxide (45%) | 2.62 | 2.62 |
| Ascorbyl Palmitate | 2.18 | 2.18 |
| Vitamin DEK Premix | 2.12 | 2.12 |
| Vitamin Premix (FSMP Premix) | 1.24 | 1.24 |
| Tocopherol-2 (mixed | 1.04 | 1.04 |
| tocopherols) | | |
| Potassium Chloride | 0.933 | 0.933 |
| Vitamin E (RRR-d-alpha) | 0.698 | 0.698 |
| tocopheryl acetate | | |
| Taurine | 0.680 | 0.680 |
| L-Carnitine | 0.457 | 0.457 |
| Ferrous Sulfate | 0.210 | 0.210 |
| Manganese Sulfate | 0.0450 | 0.0450 |
| Vitamin A Palmitate | 0.0185 | 0.0185 |
| Cupric Sulfate | 0.0078 | 0.0078 |
| Folic Acid | 0.00371 | 0.00371 |
| Chromium Chloride | 0.00231 | 0.00231 |
| Sodium Selenate | 0.00130 | 0.00130 |
| Sodium Molybdate | 0.00110 | 0.00110 |
| Zinc Sulfate | 0.00110 | 0.00110 |

### Example 7

Example 7 illustrates the sensory performance of exemplary embodiments of a liquid nutritional composition containing an oxidizable component and a water-soluble rosemary extract, as compared to a sample of a liquid nutritional composition containing an oxidizable component but with no water-soluble rosemary extract.

Four separate 100 pound batches of liquid nutritional composition were prepared with a master protein slurry, a master carbohydrate slurry, and two oil systems. The first oil system (“oil system 1”) contained marine oil and no antioxidant system. The second oil system (“oil system 2”) contained marine oil and an antioxidant system comprising ascorbyl palmitate and mixed tocopherols. The components of the master protein slurry, the components of the master carbohydrate slurries, and the components of oil systems 1 and 2 are listed below in Tables 5-8, respectively.

TABLE 5
Master Protein Slurry

| Ingredient | Amount | |
|---|---|---|
| Water | 259.4 | lb |
| Sodium Caseinate | 22.7 | lb |
| Hydrolyzed Sodium Caseinate | 22.6 | lb |
| Whey Protein Concentrate | 2.8 | lb |

Total Weight = 307.51 lb
Total Solids = 46.13 lb (15%)

TABLE 6
Master Carbohydrate Slurry

| Ingredient | Amount | |
|---|---|---|
| Water | 92.27 | lb |
| Sodium Citrate | 1.14 | lb |
| Potassium Citrate | 1.63 | lb |
| Gellan Gum | 13.61 | grams |
| Potassium Chloride | 132.11 | grams |
| Micronized-Tricalcium Phosphate | 1.74 | lb |
| Magnesium Chloride | 1.4 | lb |
| Fructooligosaccharide Powder | 5.75 | lb |
| Arabic Gum | 4.25 | lb |
| Soy Fiber | 0.85 | lb |
| Maltodextrin DE18 | 76.72 | lb |
| Sucrose | 9.21 | lb |

Total Weight = 195.29 lb
Total Solids = 97.65 lb (50%)

TABLE 7
Oil System 1

| | |
|---|---|
| Ingredient | Amount |

| | | |
|---|---|---|
| Marine Oil | 1.43 | lb |
| Fractionated Coconut (MCT) Oil | 162.4 | grams |
| Canola Oil | 94.83 | grams |
| Soy Oil | 54.13 | grams |
| Lecithin | 40.5 | grams |
| Vitamin DEK Premix | 18.10 | grams |
| Vitamin E (Tocopheryl Acetate) - 1.36 IU/mg | 6.59 | grams |
| Beta Carotene 30% | 0.83 | grams |
| Vitamin A Palmitate | 0.27 | grams |
| Vitamin K | 0.0017 | grams |

Total Weight = 2.27 lb
Total Solids = 2.27 lb (99.98%)

TABLE 8
Oil System 2

| | |
|---|---|
| Ingredient | Amount |

| | | |
|---|---|---|
| Marine Oil | 5.02 | lb |
| Fractionated Coconut (MCT) Oil | 1.25 | lb |
| Canola Oil | 331.91 | grams |
| Soy Oil | 189.46 | grams |
| Lecithin | 141.75 | grams |
| Ascorbyl Palmitate | 4.21 | grams |
| Tocopherol-2 (mixed tocopherols) | 0.7 | grams |
| Vitamin DEK Premix | 63.35 | grams |
| Vitamin E (Tocopheryl Acetate) - 1.36 IU/mg | 23.07 | grams |
| Beta Carotene 30% | 2.89 | grams |
| Vitamin A Palmitate | 0.95 | grams |
| Vitamin K | 0.0059 | grams |

Total Weight = 7.94 lb
Total Solids = 7.94 lb (99.98%)

For each of Batches 1-4, about 42.4 lb of the master protein slurry was weighed out and added to a blend kettle. For Batches 2-4, an amount of a water-soluble rosemary extract was added to the master protein slurry to provide about 5.4 grams of rosmarinic acid. For Batch 1, about 39.1 lb of the master carbohydrate slurry was added to the master protein slurry, and then all of oil system 1 (about 2.3 lb) was added to the mixture. For each of Batches 2-4, about 39.1 lb of the master carbohydrate slurry was added to the master protein slurry, and then about 2.3 lb of oil system 2 was added to the mixture.

The four separate 100 pound batches of liquid nutritional composition included different antioxidant systems to investigate the ability of a water-soluble rosemary extract to improve the sensory performance of a liquid nutritional composition containing marine oil (i.e., an oxidizable component), such as by inhibiting oxidation of the marine oil and/or reducing or otherwise masking off-flavors and aromas (e.g., fishy notes). Batch 1 did not contain an antioxidant system, and Batches 2-4 each utilized a different water-soluble rosemary extract having a unique total phenolic content to rosmarinic acid ratio (TPC:RA). The antioxidant system of each batch is shown below in Table 9.

TABLE 9
Antioxidant System

| | |
|---|---|
| Batch 1 | No antioxidant system (Oil System 1) |
| Batch 2 | Water-soluble rosemary extract |
| | (~4% w/w rosmarinic acid) (~5.4 g rosmarinic acid) |
| | (TPC:RA ratio of ≈3.21:1) |
| | Ascorbyl Palmitate (1200 ppm of Oil System 2) |
| | Mixed Tocopherols (200 ppm of Oil System 2) |
| Batch 3 | Water-soluble rosemary extract |
| | (~6% w/w rosmarinic acid) (~5.4 g rosmarinic acid) |
| | (TPC:RA ratio of ≈3.16:1) |
| | Ascorbyl Palmitate (1200 ppm of Oil System 2) |
| | Mixed Tocopherols (200 ppm of Oil System 2) |
| Batch 4 | Water-soluble rosemary extract |
| | (~10% w/w rosmarinic acid) (~5.4 g rosmarinic acid) |
| | (TPC:RA ratio of ≈1.21:1) |
| | Ascorbyl Palmitate (1200 ppm of Oil System 2) |
| | Mixed Tocopherols (200 ppm of Oil System 2) |

A sample from each batch was analyzed for intensity of various tastes and characteristics, including for example: sweet, salt, sour, bitter, vanilla, fishy, cooked milky caseinate, metallic, orange, grape/herbal, mouth cooling, corn amine, drying, and sticky mouthfeel. In a first sensory evaluation, five trained descriptive flavor panelists tasted samples from each batch that had been stored for approximately 3 months. In a second sensory evaluation, four trained descriptive flavor panelists tasted samples from each batch that had been stored for approximately 7 months. All samples were at room temperature (e.g., about 20-22° C.) when tasted. After tasting, each panelist assessed the flavor attributes of each sample using a scoring system set forth in Table 10 below and the results were then averaged. The results are shown in Table 11 for the samples stored for 3 months and Table 12 for the samples stored for 7 months.

TABLE 10

| Scale | Description |
|---|---|
| )( | Threshold |
| ½ | Very Slight |
| 1 | Slight |
| 1½ | Slight to Moderate |
| 2 | Moderate |
| 2½ | Moderate to Strong |
| 3 | Strong |

TABLE 11

| | Batch 1 | Batch 2 | Batch 3 | Batch 4 |
|---|---|---|---|---|
| Sweet | 1½ | 1½ | 1½ | 1½ |
| Salt | 1 | 1 | 1 | 1 |
| Sour | ½ | ½ | ½ | ½ |
| Bitter | 1 | ½ | ½ | ½ |
| Vanilla | 1 (creamy) | 1 (creamy) | 1 (creamy, | 1 (creamy) |
| | | | pineapple) | |
| Fishy | 1½ | ½ | ½ | 1 |
| Cooked Milky | 1 | 1 | 1 | 1 |
| Casemate | | | | |
| Metallic | ½ | ½ | ½ | ½ |
| Orange | ½ (peel) | — | — | ½ |
| Grape/Herbal | — | 1 | — | — |
| Mouth Cooling | — | ½ | — | — |
| Corn Amine | ½ | ½ | ½ | ½ |
| Drying | 1½ | 1½ | 1½ | 1½ |
| Sticky | 1½ | 1½ | 1½ | 1½ |
| Mouthfeel | | | | |

TABLE 12

| | Batch 1 | Batch 2 | Batch 3 | Batch 4 |
|---|---|---|---|---|
| Sweet | 1½ | 1½ | 1½ | 1½ |
| Salt | 1 | 1 | 1 | 1 |
| Sour | ½ | ½ | ½ | ½ |
| Bitter | 1 | ½ | ½ | ½ |
| Vanilla | 1 (creamy) | 1 (creamy) | 1 (creamy, | 1 (creamy) |
| | | | pineapple) | |
| Fishy | 1½ | ½ | ½ | 1 |
| Cooked Milky | 1 | 1 | 1 | 1 |
| Caseinate | | | | |
| Metallic | 1 | ½ | ½ | ½ |
| Orange | — | — | — | ½ |
| Grape/Herbal | — | 1½ | ½ | — |
| Mouth Cooling | — | ½ | — | — |
| Black Tea | — | 1 | — | — |
| Corn Amine | ½ | ½ | ½ | ½ |
| Drying | 1½ | 1½ | 1½ | 1½ |
| Sticky | 1½ | 1½ | 1½ | 1½ |
| Mouthfeel | | | | |

The results of the sensory evaluations indicate that batches of the liquid nutritional composition containing a water-soluble rosemary extract (Batches 2-4) exhibited less fishy notes as compared to the liquid nutritional composition with no antioxidant system (Batch 1, control). In particular, the liquid nutritional compositions utilizing a water-soluble rosemary extract having a TPC:RA ratio of at least 3.16:1 (i.e., Batches 2 and 3) reduced the fishy notes score by a full point from 1½ (Batch 1, control) to ½ over a 3 month storage time and a 7 month storage time. In addition, the liquid nutritional composition utilizing a water-soluble rosemary extract having a TPC:RA ratio of about 1.21:1 was effective for reducing the fishy note score by ½ point from 1½ (Batch 1, control) to 1 over a 3 month storage time and a 7 month storage time.

The sensory evaluations also indicate that batches of the liquid nutritional composition containing a water-soluble rosemary extract (Batches 2-4) exhibited less bitter notes over time as compared to the liquid nutritional composition with no antioxidant system (Batch 1, control). For example, after 3 months and 7 months of storage, the liquid nutritional compositions utilizing a water-soluble rosemary extract (Batches 2-4) reduced the bitter notes score by one-half point from a score of 1 (Batch 1, control) to a score of ½. Also, the sensory evaluations demonstrate the batches of the liquid nutritional composition containing a water-soluble rosemary extract (Batches 2-4) is effective for protecting against the development of metallic notes over time. For example, the liquid nutritional composition with no antioxidant system (Batch 1, control) exhibited an increase in metallic notes from a score of ½ after 3 months of storage to a score of 1 after 7 months of storage, whereas the batches of the liquid nutritional composition containing a water-soluble rosemary extract (Batches 2-4) maintained the same level of metallic notes (a score of ½) over the same time periods.

As seen from the results of the sensory evaluation, a water-soluble rosemary extract provides flavor and aroma protection to a nutritional composition including an oxidizable component. Moreover, the results indicate that the TPC:RA ratio of the water-soluble rosemary extract is an important factor in providing the flavor and aroma protection, and not simply the amount of rosmarinic acid provided by the water-soluble rosemary extract.

### Example 8

Example 8 illustrates the sensory performance of exemplary embodiments of a powder nutritional composition containing an oxidizable component and a water-soluble plant extract, as compared to a sample of a powder nutritional composition containing an oxidizable component but with no water-soluble plant extract.

Four separate batches of powder nutritional composition were prepared with a master protein slurry, a master oil system, and a carbohydrate-mineral system. The oil system contained an antioxidant system comprising ascorbyl palmitate and mixed tocopherols. The components of the master protein slurry, the components of the master oil system, and the components of the carbohydrate-mineral system are listed below in Tables 13-15, respectively.

TABLE 13
Master Protein Slurry

| Ingredient | Amount | |
|---|---|---|
| Water | 398.6 | lb |
| Calcium Caseinate | 52.73 | lb |
| Milk Protein Concentrate (80%) | 58.25 | lb |
| Whey Protein Concentrate | 6.6 | lb |

Total Weight = 516.13 lb
Total Solids = 113.55 lb (22%)

TABLE 14
Master Oil System

| Ingredient | Amount | |
|---|---|---|
| Marine Oil | 24.9 | lb |
| Soy Oil | 38.1 | lb |
| Lecithin | 2.62 | lb |
| Vitamin DEK Premix | 384.63 | g |
| Vitamin E (Tocopheryl Acetate) - 1.36 IU/mg | 126.74 | g |
| Vitamin A Palmitate | 3.37 | g |
| Tocopherol-2 (Mixed Tocopherols) | 186.9 | g |
| Ascorbyl Palmitate | 392.85 | g |

Total Weight = 68.04 lb
Total Solids = 68.02 lb (99.97%)

TABLE 15
Carbohydrate-Mineral System

| Ingredient | Amount | |
|---|---|---|
| Potassium Citrate | 1.33 | lb |
| Calcium Carbonate | 178.10 | g |
| Magnesium Phosphate Dibasic | 1.00 | lb |
| Maltodextrin DE18 | 29.02 | lb |
| Sucrose | 9.19 | lb |

The four separate Batches were labeled as Batches 301-304. For each of Batches 301-304, about 103.2 lb of the master protein slurry was weighed out and added to a blend kettle. The carbohydrate-mineral system was added to each of Batches 301-304. For Batches 302 and 304, an amount of a water-soluble rosemary extract was added to the combined master protein slurry and carbohydrate-mineral system to provide about 6.9 grams of rosmarinic acid. For Batch 303, an amount of a water-soluble spearmint extract was added to the combined master protein slurry and carbohydrate-mineral system to provide about 10.4 grams of rosmarinic acid. After sufficient mixing and a brief hold period, about 13.6 lb of the master oil system was added to each of Batches 301-304. A vitamin C solution and a water-soluble vitamin solution was added to each of Batches 301-304, and a flavor solution was added to Batch 304 (whereas the flavor was dryblended in Batches 301-303). The Batches were then subjected to ultra-high temperature processing (“UHT”) and subsequently spray dried.

The four separate batches of powder nutritional composition included different antioxidant systems to investigate the ability of a water-soluble plant extract to improve the sensory performance of a powder nutritional composition containing marine oil (i.e., an oxidizable component), such as by inhibiting oxidation of the marine oil and/or reducing or otherwise masking off-flavors and aromas (e.g., fishy notes). Batch 301 only contained the antioxidant system used in the master oil system, while Batches 2 and 4 each utilized a water-soluble rosemary extract, and Batch 3 utilized a water-soluble spearmint extract. The antioxidant system of each batch is shown below in Table 16.

TABLE 16
Antioxidant System

| | |
|---|---|
| Batch 301 | Ascorbyl Palmitate (13,200 ppm of Master Oil System) |
| | Mixed Tocopherols (6,280 ppm of Master Oil System) |
| Batch 302 | Water-soluble rosemary extract |
| | (~6% w/w rosmarinic acid) (~6.9 g rosmarinic acid) |
| | (TPC:RA ratio of ≈3.16:1) |
| | Ascorbyl Palmitate (13,200 ppm of Master Oil System) |
| | Mixed Tocopherols (6,280 ppm of Master Oil System) |
| Batch 303 | Water-soluble spearmint extract |
| | (~5% w/w rosmarinic acid) (~10.4 g rosmarinic acid) |
| | (TPC:RA ratio of ≈2:1) |
| | Ascorbyl Palmitate (13,200 ppm of Master Oil System) |
| | Mixed Tocopherols (6,280 ppm of Master Oil System) |
| Batch 304 | Water-soluble rosemary extract |
| | (~6% w/w rosmarinic acid) (~6.9 g rosmarinic acid) |
| | (TPC:RA ratio of ≈3.16:1) |
| | Ascorbyl Palmitate (13,200 ppm of Master Oil System) |
| | Mixed Tocopherols (6,280 ppm of Master Oil System) |

A sample from each batch was reconstituted and analyzed for intensity of various tastes and characteristics, including for example: sweet, sour, salt, bitter, vanilla, cooked milk, citrus (lemon), herbal/medicinal, chemical/solventy, fishy, drying, and powdery mouthfeel. In a first sensory evaluation, four trained descriptive flavor panelists tasted samples from each batch that had been stored for approximately 4 months. After tasting, each panelist assessed the flavor attributes of each sample using a scoring system set forth in Table 17 below and the results were then averaged. The results are shown in Table 18.

TABLE 17

| Scale | Description |
|---|---|
| )( | Threshold |
| ½ | Very Slight |
| 1 | Slight |
| 1½ | Slight to Moderate |
| 2 | Moderate |
| 2½ | Moderate to Strong |
| 3 | Strong |

TABLE 18

| | Batch 301 | Batch 302 | Batch 303 | Batch 304 |
|---|---|---|---|---|
| Sweet | 1½ | 1½ | 1½ | 1½ |
| Sour | 1 | 1 | 1 | 1 |
| Salt | 1 | 1 | 1 | 1 |
| Bitter | 1 | 1 | 1 | 1 |
| Vanilla | 1½ | 1 | 1 | 1½ |
| (buttery/fruity) | | | | (buttery) |
| Cooked Milk | 1 | 1 | 1 | 1 |
| Citrus (lemon) | 1 | 1 | 1 | — |
| Herbal/Medicinal | — | 1 | 1 | 1 |
| Chemical/Solventy | — | — | 1 | — |
| Fishy | ½ | — | — | — |
| Drying | 1½ | 1½ | 1½ | 1½ |
| Powdery Mouthfeel | 1 | 1 | 1 | 1 |
| Mouthfeel Comments | airy, | airy, | airy, | airy, |
| | thick | thick | thick | thick |
| | mouthfeel | mouthfeel | mouthfeel | mouthfeel |
| % Oxygen Headspace | 0.1 | 0.0 | 0.1 | 1.4 |

The results of the sensory evaluations indicate that batches of the powder nutritional composition containing a water-soluble plant extract comprising rosmarinic acid (Batches 2-4) exhibited less fishy notes as compared to the powder nutritional composition with no water-soluble plant extract (Batch 301, control). In particular, the powder nutritional compositions utilizing a water-soluble plant extract comprising rosmarinic acid reduced the fishy notes score by one-half point from ½ (Batch 1, control) to 0 (not detected) over a 4 month storage time.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. Also, to the extent that the term “in” or “into” is used in the specification or the claims, it is intended to additionally mean “on” or “onto,” respectively. Furthermore, to the extent the term “connect” is used in the specification or claims, it is intended to mean not only “directly connected to” but also “indirectly connected to” such as connected through another component or components.

While the present application has been illustrated by the description of embodiments thereof, and while the embodiments have been described in considerable detail, it is not the intention of the Applicants to restrict or in any way limit the scope of the appended claims to such detail. Additional advantages and modifications will readily appear to those skilled in the art. Therefore, the application, in its broader aspects, is not limited to the specific details, the representative compositions and processes, and illustrative examples shown and described. Accordingly, departures may be made from such details without departing from the spirit or scope of the general inventive concepts.

1. A nutritional composition comprising:
(a) a protein;
(b) a carbohydrate;
(c) a fat; and
(d) a water-soluble plant extract comprising rosmarinic acid and having a total phenolic content;
wherein at least one of the protein, the carbohydrate, and the fat includes an oxidizable component; and
wherein a ratio of the total phenolic content to the rosmarinic acid ranges from about 1.1:1 to about 3.5:1.

2. The nutritional composition of claim 1, wherein the rosmarinic acid comprises from about 0.0004 wt % to about 0.07 wt % of the nutritional composition.

3. The nutritional composition of claim 1, wherein the total phenolic content is from about 1 mg to about 600 mg of gallic acid equivalents per serving of the nutritional composition, wherein a serving is 237 mL.

4. The nutritional composition of claim 1, wherein the water-soluble plant extract comprises a water-soluble rosemary extract.

5. The nutritional composition of claim 1, wherein the water-soluble plant extract comprises a water-soluble spearmint extract.

6. The nutritional composition of claim 1, wherein the protein is about 1 wt % to about 30 wt % of the nutritional composition, the carbohydrate is about 5 wt % to about 25 wt % of the nutritional composition, and the fat is about 0.5 wt % to about 20 wt % of the nutritional composition.

7. The nutritional composition of claim 1, wherein the protein is selected from the group consisting of whey protein concentrates; whey protein isolates, whey protein hydrolysates, acid caseins, sodium caseinates, calcium caseinates, potassium caseinates, casein hydrolysates, milk protein concentrates, milk protein isolates, milk protein hydrolysates, nonfat dry milk, condensed skim milk, soy protein concentrates, soy protein isolates, soy protein hydrolysates, pea protein concentrates, pea protein isolates, pea protein hydrolysates, collagen proteins, collagen protein isolates, rice proteins, potato proteins, earthworm proteins, insect proteins, and combinations thereof.

8. The nutritional composition of claim 1, wherein the carbohydrate is selected from the group consisting of maltodextrin, hydrolyzed starch, glucose polymers, corn syrup, corn syrup solids, rice-derived carbohydrates, sucrose, glucose, fructose, lactose, high fructose corn syrup, honey, sugar alcohols, isomaltulose, sucromalt, puilulan, potato starch, corn starch, fructooligosaccharides, galactooligosaccharides, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy, pectin, high methoxy pectin; cereal beta-glucans, carrageenan, psyllium, and combinations thereof.

9. The nutritional composition of claim 1, wherein the fat is selected from the group consisting of coconut oil, fractionated coconut oil, medium chain triglycerides (MCT) oil; palm oil, palm kernel oil, palm olein, and combinations thereof.

10. The nutritional composition of claim 1, wherein the fat includes an oxidizable component and the oxidizable component comprises an unsaturated fatty acid.

11. The nutritional composition of claim 10, wherein the fat including an oxidizable component is at least one of marine oil, algal oil, canola oil, flaxseed oil, borage oil, safflower oil, high oleic safflower oil, high gamma-linolenic acid (GLA) safflower oil, corn oil, soy oil, sunflower oil, high oleic sunflower oil, and cottonseed oil.

12. The nutritional composition of claim 10, wherein the oxidizable component comprises an omega-3 polyunsaturated fatty acid selected from the group consisting of eicosapentaenoic acid, docosahexaenoic acid, alpha-linolenic acid, and combinations thereof.

13. The nutritional composition of claim 12, wherein the omega-3 polyunsaturated fatty acid comprises eicosapentaenoic acid and docosahexaenoic acid.

14. The nutritional composition of claim 13, wherein a weight ratio of eicosapentaenoic acid to docosahexaenoic acid is about 1:5 to about 5:1.

15. The nutritional composition of claim 10, wherein the oxidizable component comprises an omega-6 polyunsaturated fatty acid selected from the group consisting of gamma-linolenic acid, linoleic acid, dihomo-gamma-linolenic acid, arachidonic acid, and combinations thereof.

16. The nutritional composition of claim 1, further comprising at least one antioxidant selected from the group consisting of ascorbic acid, ascorbyl palmitate, retinyl palmitate, tocopherols, ascorbate salts, carotenoids, oil-soluble rosemary extract, and combinations thereof.

17. The nutritional composition of claim 1, wherein the nutritional composition is a liquid and has a caloric density of about 0.5 kcal/mL to about 3 kcal/mL.

18. The nutritional composition of claim 1, wherein the nutritional composition is a powder, and the rosmarinic acid comprises from about 0.002 wt % to about 0.35 wt % of the nutritional composition.

19. The nutritional composition of claim 1, wherein the nutritional composition is in the form of an aqueous emulsion having a continuous aqueous phase and a discontinuous non-aqueous phase.

20. A method of reducing off-flavors and aromas in a liquid nutritional composition, the method comprising:
(a) preparing an aqueous protein slurry comprising at least one protein and a water-soluble plant extract, wherein the water-soluble plant extract is extracted with a solvent system having an octanol-water partition coefficient of no more than 0.5;
(b) preparing an aqueous carbohydrate slurry comprising at least one carbohydrate;
(c) preparing an oil blend comprising a fat having an oxidizable component; and
(d) mixing together the aqueous protein slurry, the aqueous carbohydrate slurry, and the oil blend to form the liquid nutritional composition.

21. The method of claim 20, wherein the water-soluble plant extract comprises a water-soluble rosemary extract.

22. The method of claim 20, wherein the water-soluble plant extract comprises a water-soluble spearmint extract.

23. The method of claim 20, further comprising heat treating the liquid nutritional composition and homogenizing the liquid nutritional composition.

24. The method of claim 20, wherein the solvent system comprises ethanol.

25. The method of claim 20, wherein the solvent system comprises acetone.

26. The method of claim 21, wherein the water-soluble rosemary extract comprises rosmarinic acid and has a total phenolic content, and wherein a ratio of the total phenolic content to the rosmarinic acid ranges from about 1.1:1 to about 3.5:1.

27. The method of claim 21, wherein the rosmarinic acid is about 4 wt % to about 20 wt % of the water-soluble rosemary extract.

28. The method of claim 20, wherein the at least one protein is selected from the group consisting of whey protein concentrates, whey protein isolates, whey protein hydrolysates, acid caseins, sodium caseinates, calcium caseinates, potassium caseinates, casein hydrolysates, milk protein concentrates, milk protein isolates, milk protein hydrolysates, nonfat dry milk, condensed skim milk, soy protein concentrates, soy protein isolates, soy protein hydrolysates, pea protein concentrates, pea protein isolates, pea protein hydrolysates, collagen proteins, collagen protein isolates, rice proteins, potato proteins, earthworm proteins, insect proteins, and combinations thereof.

29. The method of claim 20, wherein the at least one carbohydrate is selected from the group consisting of maltodextrin, hydrolyzed starch, glucose polymers, corn syrup, corn syrup solids, rice-derived carbohydrates, sucrose, glucose, fructose, lactose, high fructose corn syrup, honey, sugar alcohols, isomaltulose, sucromalt, pullulan, potato starch, corn starch, fructooligosaccharides, galactooligosaccharides, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy, pectin, high methoxy pectin, cereal beta-glucans, carrageenan, psyllium, and combinations thereof.

30. The method of claim 20, wherein the fat having an oxidizable component is at least one of marine oil, algal oil, canola oil, flaxseed oil, borage oil, safflower oil, high oleic safflower oil, high gamma-linolenic acid (GLA) safflower oil, corn oil, soy oil, sunflower oil, high oleic sunflower oil, and cottonseed oil.

31. The method of claim 20, wherein the oxidizable component comprises an omega-3 polyunsaturated fatty acid selected from the group consisting of eicosapentaenoic acid, docosahexaenoic acid, alpha-linolenic acid, and combinations thereof.

32. The method of claim 31, wherein the omega-3 polyunsaturated fatty acid comprises eicosapentaenoic acid and docosahexaenoic acid.

33. The method of claim 32, wherein a weight ratio of eicosapentaenoic acid to docosahexaenoic acid is about 1:5 to about 5:1.

34. The method of claim 20, wherein the oxidizable component comprises an omega-6 polyunsaturated fatty acid selected from the group consisting of gamma-linolenic acid, linoleic acid, dihomo-gamma-linolenic acid, arachidonic acid, and combinations thereof.

35. The method of claim 20, wherein the oil blend further comprises at least one of coconut oil, fractionated coconut oil, medium chain triglycerides (MCT) oil, palm oil, palm kernel oil, and palm olein.
